(19) United States
(12) Patent Application Publication
(10) Pub. No.: US 2012/0283379 A1
(43) Pub. Date: Nov. 8, 2012

(54) SILICA PARTICLE INCLUDING A MOLECULE OF INTEREST, METHOD FOR PREPARING SAME AND USES THEREOF

(75) Inventors: Aurélien Auger, Le Mans (FR); Olivier Raccurt, Chelieu (FR); Olivier Jean Christian Poncelet, Grenoble (FR); Jorice Samuel, Vizille (FR); Sylvie Sauvaigo, Grenoble (FR); Chloé Schubert, Tignes (FR)

(73) Assignee: Commissariat a l'energie atomique et aux energies alternatives, Paris (FR)

(21) Appl. No.: 13/508,698

(22) PCT Filed: Nov. 10, 2010
(86) PCT No.: PCT/EP2010/067190

§ 371 Date: Jul. 30, 2012

(30) Foreign Application Priority Data
Nov. 10, 2009 (FR) 09 57920

(51) Int. Cl. C07H 23/00; C08F 120/06; C08L 33/02; C09K 3/00; C08F 8/42; B82Y 15/00; B82Y 30/00; B82Y 40/00

(52) U.S. Cl. 524/556; 536/23.1; 252/183.11; 525/329.7; 525/342; 525/54.2; 977/773; 977/795; 977/896

(57) ABSTRACT

What is provided includes a nanoparticle of porous silica, incorporating at least one molecule of interest, the silica network inside said nanoparticle being functionalized by at least one group capable of setting up an ionic and/or hydrogen non-covalent bond with the molecule of interest, whereby the molecule(s) of interest is(are) linked to the silica network solely by non-covalent bonds. In addition, a method for preparing said silica particle and uses thereof is provided.

### TECHNICAL FIELD

The present invention concerns the field of silica particles, and more particularly silica particles capable of incorporating, encapsulating or confining molecules of interest such as biomolecules.

The present invention therefore proposes a silica particle incorporating, encapsulating or confining a molecule of interest. For the present invention, advantage is taken of the presence within a silica particle of groups capable of setting up a non-covalent bond, of electrostatic interaction and/or hydrogen bridge type, with said molecule of interest.

The present invention also concerns a method for preparing said silica particle i.e. a method for incorporating, encapsulating or confining a molecule of interest in a silica particle and the uses of said particle in particular for combatting infringement or for biological analysis.

### STATE OF THE PRIOR ART

The synthesis of silica nanoparticles via sol-gel route using silicon alkoxides has been described in the literature since the work by Stöber at the end of the 60's who developed a method carrying his name and which can be used to obtain micrometric silica particles [1].

On the basis of this method, numerous developments have allowed the size of the particles to be reduced down to nanoparticle size ([2], [3]). The synthesis method via Stöber route uses a silicon alkoxide such as tetraethoxysilane (TEOS) or tetramethoxysilane (TMOS) in a solution of alcohol and water. The solution is then heated to obtain hydrolysis of the silicon alkoxide, then the addition of a certain quantity of catalyst, here HCl, allows condensation of the silica into the form of particles.

Another synthesis mode based on a sol-gel reaction in an emulsion make it possible to obtain particles of smaller size and more monodispersed than with the Stöber method. This method is called sol-gel synthesis by reverse microemulsion ([4]-[6]).

With this method, an emulsion of water in an oil phase is formed by means of a surfactant, and in some cases a co-surfactant. The size of the micelles is generally nanometric, they therefore form a nanoreactor in which the hydrolysis-condensation reaction of the silicon alkoxide takes place. It is the size of the micelle which determines the size of the formed particle.

25

With the Stöber method, the encapsulation of an organic molecule requires covalent grafting between this molecule and the silica network. Therefore, for this synthesis route a certain number of organic fluorophores were incorporated in the silica particles. This requires the creation of a silicon alkoxide covalently bonded to the organic molecule by reaction between an isothiocyanate function and an amine function. Numerous examples are given in the literature with FITC (fluorescein isothiocyanate) or RBITC (Rhodamine B isothiocyanate) encapsulated in silica using the Stöber method [7].

In these methods, the fluorophore having an isothiocyanate function is mixed with aminopropyl-triethoxysilane (APTES). The isothiocyanate function reacts with the amine function of the alkoxide and forms a fluorescent silicon alkoxide which is then added to the synthesis within a certain proportion relative to the main silicon alkoxide which is TEOS. In general, this concentration is of the order of 1 to 5%. However, this concentration may be higher and may reach 25%. If the fluorescent alkoxide obtained by reaction between the isothiocyanate function and APTES is added at the start of the reaction, the fluorophore is distributed throughout the particle.

This route has also been used to form structures of core/shell type either with the core comprising the organic molecule and the surface comprising the silica or conversely [8]. In this case, the method is conducted in two identical successive steps.

This encapsulation method by covalent grafting also functions for non-fluorescent molecules following the same procedure: reaction between a silicon alkoxide (APTES) and the molecule to be covalently linked with the silica ([9], [10]). It is also possible to obtain a silica core and a shell containing the organic molecules by covalent bonding or by sol-gel reaction ([7], [11], [12]).

Regarding syntheses via micellar route, the encapsulation method of organic molecules by covalent grafting, as for the previously described Stöber method, also functions but it is not necessarily required. Since there is a micellar medium, the confinement of the molecules or particles in the micelle and therefore their encapsulation with silica are possible.

The research work conducted in the Applicant's laboratory has shown that this method allows the encapsulation of fluorescent organic molecules such as rhodamine and fluorescein [13]. This encapsulation method works for molecules soluble in the aqueous phase. In this method, after the formation of the reverse emulsion, the first step consists in adding the molecule to be encapsulated which, if it has higher solubility in water than in the oil phase, will essentially concentrate inside the micelles. The silicon alkoxide (TEOS) and the catalyst for forming the silica particles are then added. The formation of the silica particle takes place from outside the micelle towards the inside thereby encapsulating the molecule present in the micelle. The silica particles thus obtained are then functionalized through the addition of coupling agents such as N-2-(aminoethyl)-3-aminopropylthiethoxysilane.

Work conducted in the Applicant's laboratory has shown that in relation to the solubility of the organic molecule, the molecule if it is highly soluble in water will come to be confined in the core of the silica particle. If it has lower solubility, it will either be distributed into the particle or will lie more on the surface. If it is not at all water-soluble and if it does not form a covalent bond with the silica, it is not incorporated in the particle.

With respect to deoxyribonucleic acid (DNA) and biological macromolecules, encapsulation strategies and methods have been described in the literature. Biomacromolecules have already been successfully encapsulated in objects of micrometric and nanometric size dispersed in water (hydrophilic objects).

Biomacromolecules such as bovine serum albumin (BSA) and DNA have been successfully encapsulated in hollow silica microcapsules [14]. These microcapsules are synthesized by dual micellar route (water-oil-water). The objects obtained are of micrometric size. The same encapsulation format was developed via conventional sol-gel route, the Stöber method. The presence of DNA in the objects was detected by fluorescence by means of DNA intercalants of ethidium bromide type (EtBr). A similar method described in the literature describes the synthesis of silica nanoparticles by sol-gel route having smaller diameters: ˜200 nm [15].

Different nanoparticles of silica (˜40 nm) have been synthesized using reverse microemulsion with TEOS in which, in the core part of these nanoparticles, there are biomolecules conjugated with the Cy5 fluorophore (cyanine fluorescing in the red 650-670 nm) [16]. The studied molecules conjugated with Cy5 were polylysine (70000-15000 g.mol⁻¹), immunoglobins G (IgG; 150000 g.mol⁻¹), BSA (68000 g.mol⁻¹), insulin (5700 g.mol⁻¹) and pepsin (42500 g.mol⁻¹). The presence of the biomolecular core was detected by fluorescence emitted by the Cy5. In addition, it is important to point out that these biomolecules have characteristics of weight and size smaller than those of plasmidic DNA that is particularly used in the present invention i.e. DNA containing 3000 base pairs (bp).

The use of a synthesis method via micellar route for encapsulation of DNA in polymer particles has already been published [17]. The article by Hammady et al. gives as example the encapsulation of DNA in beads of PVA (PolyVinyl Acrylic) and PLA (polylactide). In this case, the DNA in principle lies inside the spheres. However, it is neither accessible for analysis nor usable for as long as the polymer is not destroyed by dissolution. Therefore, the difference compared with the present invention described below concerns firstly the DNA encapsulating material (polymer), and secondly the need for dissolution of this material in order to access the DNA.

It is also possible to cite other types of DNA-encapsulating particles or [symbol] containers [symbol] . The article by Cisse et al. describes the encapsulation of DNA in capsules whose wall is formed of a lipid membrane [18]. This is composed of a double surfactant layer. This type of particle does not have much in common with a silica particle according to the present invention since it is a kind of bubble with a liquid inside, composed of a thin wall formed by a double molecular layer.

A third approach consists in functionalizing the surface of the nano-objects so as to anchor the desired biomolecules thereupon ([19], [20]). In these documents, the preparation and functionalization steps of the nanoparticles are distinct and are conducted prior to contacting with the biomolecules.

In general, the surface of the nano-objects is functionalized by amino groups which, via electrostatic interactions, bind with the biomolecules. The nanoparticles have a positive surface charge by means of the amino groups. Therefore, enriching with DNA is performed in the form of [symbol] DNA-nanoparticle [symbol] complexes which involve electrostatic links between the amino groups of the nanoparticles and the negative charge of the DNA phosphate groups. Similar operating modes have been developed consisting in improving the complexing with DNA. For example, a regular porosity has been generated in silica nanoparticles in order to accommodate the proximity of the biomolecule and better in order to promote the electrostatic bond with the functionalized surface of the silica nanoparticles [21].

One last route is used for encapsulating DNA or biomolecules in silica obtained by sol-gel in film form. This route does not afford particles but films. Biomolecules were stored in inorganic gels or hybrid organic-inorganic gels. The encapsulation of DNA (50 bp) was conducted in hybrid polyvinyl alcohol-silica gels. Analysis of small DNA fragments encapsulated in gels has shown that complexing probably takes place between the silica network and the phosphate groups. This explains why most DNA molecules have not been able to be extracted from gels [22].

There is therefore a true need for silica particles such as silica nanoparticles capable of incorporating or encapsulating a molecule of interest and in particular a molecule of interest of large size such as DNA, providing protection of the molecule of interest whilst leaving it accessible to the elements and constituents of the outside medium. In addition, such particles must be able to be prepared using a method easy to implement, not requiring the preparation of reaction precursors.

### DISCLOSURE OF THE INVENTION

The present invention makes it possible to overcome the drawbacks and technical problems listed above. Indeed, it proposes particulate, spherical, silica-based materials that in particular are nanoparticulate, porous, incorporating molecules of interest, in which the molecules of interest are held within the particles whilst remaining accessible to elements present outside the particles such as enzymes, and without implementing any covalent bonding between the molecules of interest and the particles. Despite their confinement, the molecules of interest maintain their chemical reactivity. On this account the molecules present outside the particles, via the porosity of the particles, are able to access the molecules of interest and to react therewith. In addition, said materials can be prepared using a method applicable on industrial scale, not requiring any cumbersome procedures or steps and using products that are easily accessible.

The work by the inventors has evidenced that the combined use of a silicon alkoxide, also called [symbol] silane-based compound [symbol] , forming a complete network, and of a silicon alkoxide having at least one group capable of setting up a non-covalent bond with the molecule of interest allows the fabrication of silica particles such as silica nanoparticles incorporating or confining molecules of interest. Indeed, the silica particles obtained with said method are functionalized, inside the silica network, by groups capable of setting up a non-covalent bond. This specific functionalization allows non-covalent bonds of electrostatic or hydrogen-bridge type with the molecule of interest.

Therefore, for example, when the molecule of interest is DNA, this latter composed of phosphates linked to sugars which themselves are linked to nitrogen-containing bases has a negative zeta potential. The use of a silicon alkoxide having one or more amino groups and a silicon alkoxide forming a complete network allows the obtaining of silica particles functionalized by amino groups inside the silica network. The negatively charged phosphate comes to interact electrostatically (or via hydrogen bridge) with the amino groups of the silica network. At the time of hydrolysis of silicon derivatives i.e. silicon alkoxides, the DNA is confined in the silica particle and in particular in the core of the silica particle, which is chiefly due to the electrostatic bonds and hydrophilic properties of DNA. The DNA is then encapsulated in the porous silica network of the particles and particularly in the centre of said network. A block diagram of the final structure of the particle is given in FIG. 1.

It is to be noted that the silica particles according to the present invention are not hollow particles. These particles may appear as particles of core/shell type, in particular when analysed under transmission electron microscopy. However, these particles are not [symbol] true [symbol] particles of core/shell type since they do not have a core with a 1^{st} chemical compound and a shell with a 2^{nd} chemical compound, the two compositions being separate, and they do not either have a materialized interface between the core and the shell. However, for brevity, in the present invention we use the term [symbol] core [symbol] to define the centre of the particle comprising nearly all the molecules of interest and the term [symbol] shell [symbol] to define the outer part of the particle not comprising any molecule of interest.

The encapsulated molecules of interest are protected by the silica. The silica is therefore used as stabilizing support for the molecule of interest. However, the method of the invention allows a silica shell to be obtained surrounding the core of the particle containing the molecule of interest. This shell is sufficiently porous to make the molecule of interest accessible for the constituents present in the outside medium, whether for small molecules such as fluorescent nucleotides or for larger molecules such as enzymes.

Additionally, since the encapsulation according to the invention uses non-covalent bonds, the properties of the molecule of interest such as activity and/or recognition are not modified.

The present invention therefore concerns a porous silica particle incorporating at least one molecule of interest and essentially obtained from the hydrolysis of:
at least one first silicon alkoxide of formulas Si(OR₁)₄, R₂Si(OR₃)₃ or R₄R₅Si(OR₆)₂ where R₁, R₃ and R₆, the same or different, are an alkyl radical with to 6 carbon atoms and R₂, R₄ and R₅, the same or different, represent a hydrogen, an alkyl radical with 1 to 6 carbon atoms or an alkenyl radical with 1 to 6 carbon atoms; and
at least one second silicon alkoxide having at least one group capable of setting up an ionic and/or hydrogen non-covalent bond with the molecule of interest.

More particularly, the present invention concerns a silica particle comprising at least one molecule of interest, the silica network inside the said particle being functionalized by at least one group capable of setting up an ionic and/or hydrogen non-covalent bond with the molecule of interest, whereby the molecule(s) of interest are linked to the silica network solely by non-covalent bonds.

Advantageously, the silica particle of the invention has a silica network functionalized by several groups capable of setting up an ionic and/or hydrogen non-covalent bond with the molecule of interest. These groups are distributed within the silica particle in the form of a decreasing gradient from the centre of the particle towards outside the particle, no group being present on the surface of the particle.

By [symbol] silica particle incorporating at least one molecule of interest [symbol] in the present invention is meant a silica particle inside which there is at least one molecule of interest. Under the present invention, the molecule of interest, or group of molecules of interest, does not lie on the surface of the silica particle.

Advantageously, the silica particle of the invention is a particle of core/shell type in the core of which there is at least one molecule of interest. When the silica particle incorporates several molecules of interest, the same or different, they lie for the most part inside the particle and in particular in the core of the particle.

The distribution of the molecules of interest in the silica particle may be in the form of a gradient with a strong concentration of molecules of interest at the centre of the particle and in particular at the centre of the core of this particle, and a smaller concentration on moving further away from this centre.

Therefore, at least 80%, at least 90%, at least 95%, at least 98%, at least 99% of the molecules of interest and/or group of molecules of interest lie inside and in particular in the core of the particle. The expressions [symbol] silica particle encapsulating at least one molecule of interest [symbol] , [symbol] silica particle incorporating at least one molecule of interest [symbol] or [symbol] silica particle confining at least one molecule of interest [symbol] are equivalent and can be used interchangeably.

The silica particle of the invention is of nanometric size; the term [symbol] nanoparticle [symbol] , [symbol] sphere [symbol] or [symbol] nanosphere [symbol] can thus be used. As indicated in the experimental part below, it is possible to cause the mean size of the silica particles of the invention to vary by acting on the quantities of molecules of interest and/or on the ratio of polar liquid (polar solvent such as water)/surfactant/nonpolar liquid (oil phase chiefly formed by the nonpolar or scarcely polar solvent). Advantageously, the silica particles of the invention have a mean size equal to or less than 150 nm, than 120 nm, than 100 nm, than 80 nm or than 60 nm. The silica particles of the invention may have a mean size of the order of 40 nm (i.e. 40±10 nm). Alternatively, the silica particles of the invention have a mean size of less than 40 nm, than 20 nm and in particular of the order of 10 nm (i.e. 10±5 nm). It will be obvious for persons skilled in the art that the mean size of the silica particle according to the invention will influence the size of the incorporated molecule(s) of interest.

The silica particles of the invention are porous with an open porosity and are particularly mesoporous with an open porosity. Advantageously, they have a pore size of less than 100 angstroms and a pore size distribution ranging from 1 to 100 angstroms, in particular from 10 to 90 angstroms, more particularly from 15 to 80 angstroms and further particularly from 20 to 70 angstroms, and a specific surface area of 200 to 900 m².g⁻¹, in particular from 300 to 800 m².g⁻¹ and more particularly from 400 to 700 m².g⁻¹.

The silica particle of the invention has a certain number of characteristics listed below:
the molecule of interest is inside the silica particle and particularly in the core of this particle, the silica acting as protector for the molecule of interest;
the molecule of interest is held inside the silica particle by means of the non-covalent bonds used, no covalent bond existing between the silica network and the molecule of interest;
since the silica particle is porous, small molecules are able to enter inside the silica particle (if the molecule of interest is a nucleic acid, an intercalant can intercalate in the nucleic acid and emit a specific signal in the presence of this acid);
the molecule of interest incorporated in the silica particle is also accessible to enzymatic reactions being for which it is the substrate, such enzymatic reactions able to lead to a detectable signal such as a change in labelling of the molecule of interest.

Therefore the molecule(s) of interest incorporated in the silica particle according to the invention is(are) held and protected inside the silica particle of the invention. The molecule(s) of interest incorporated in the silica particle of the invention is(are) accessible to molecules present in the outside medium of the silica particle, according to the invention. The molecule(s) of interest incorporated in the silica particle of the invention is(are) able to interact with these molecules.

By [symbol] molecule of interest [symbol] in the present invention is meant any molecule having at least one group able to set up an ionic and/or hydrogen non-covalent bond with the second silicon alkoxide such as defined in the present invention. This molecule may be natural or synthetic, of small or large size (macromolecule). The molecule of interest is advantageously chosen from the group consisting of an enzyme, a protein, an oligopeptide, a peptide, an antigen, an antibody, a nucleic acid, a polymer or a carbohydrate. The molecule of interest may be labelled in particular by a fluorochrome (fluorescein, Cy5, Cy3, rhodamine), a radioactive isotope, an enzyme (alkaline phosphatase, horseradish peroxidase), colloidal gold, biotin or digoxigenin.

The expression [symbol] nucleic acid [symbol] used herein is equivalent to the following terms and expressions: [symbol] polynucleotide sequence [symbol] , [symbol] nucleotide molecule [symbol] , [symbol] polynucleotide [symbol] , [symbol] nucleotide sequence [symbol] . By [symbol] nucleic acid [symbol] in the present invention is meant a chromosome; a gene; a regulatory polynucleotide; a single-strand or double-strand, genomic, chromosomal, chloroplastic, plasmidic, mitochondrial, recombinant or complementary DNA; total RNA; messenger RNA; ribosomal RNA; transfer RNA; a portion or a fragment thereof.

A DNA in the present invention may have 10 bp (or 20 nucleotides) to 5 000 bp (or 10 000 nucleotides), and in particular from 20 bp (or 40 nucleotides) to 4 000 bp (or 8 000 nucleotides).

By [symbol] ionic non-covalent bond [symbol] in the present invention is meant an intermolecular interaction between at least two positively or negatively charged groups. The expressions [symbol] ionic non-covalent bond [symbol] , [symbol] ionic interaction [symbol] , [symbol] electrostatic bond [symbol] or [symbol] electrostatic interaction [symbol] are equivalent and can be used interchangeably.

In the present invention, this intermolecular interaction is attracting. It involves a negatively charged group of the molecule of interest and a positively charged group of the second silicon alkoxide. Alternatively, it involves a positively charged group of the molecule of interest and a negatively charged group of the second silicon alkoxide.

By [symbol] hydrogen non-covalent bond [symbol] is meant a bond in which a hydrogen atom bound covalently to an atom A is attracted by an atom B containing a pair of free electrons (:B). This leads to strong polarization of the A-H bond and to electrostatic interactions between H(δ+) and :B. The expression [symbol] hydrogen non-covalent bond [symbol] is equivalent to the expression [symbol] dipole-dipole interaction [symbol] .

In the present invention, the atom A may be an atom of the second silicon alkoxide, and atom B an atom of the molecule of interest. Alternatively, atom A may be an atom of the molecule of interest and atom B an atom of the second silicon alkoxide.

In the present invention, the first silicon alkoxide of formulas Si(OR₁)₄, R₂Si(OR₃)₃ or R₄R₅Si (OR₆)₂ where R₁, R₃ and R₆, the same or different, are an alkyl radical with 1 to 6 carbon atoms and R₂, R₄ and R₅, the same or different represent a hydrogen, an alkyl radical with 1 to 6 carbon atoms or an alkenyl radical with 1 to 6 carbon atoms, mainly takes part in the formation of the silica network and in particular in the formation of the shell of the silica particle according to the invention.

Indeed, this first silicon alkoxide may advantageously have a hydrolysis rate equal to or less than the hydrolysis rate of the second silicon alkoxide.

By [symbol] alkyl radical with 1 to 6 carbon atoms [symbol] is meant a straight-chain or branched alkyl radical having 1 to 6 carbon atoms and in particular 1 to 4 carbon atoms.

By [symbol] alkenyl radical with 1 to 6 carbon atoms [symbol] is meant a straight-chain or branched alkenyl radical having at least one double bond and from 1 to 6 carbon atoms, in particular 1 to 4 carbon atoms.

Advantageously, R₂, R₄ and R₅, the same or different, are chosen from the group consisting of a hydrogen, methyl, ethyl, vinyl and propyl.

The first silicon alkoxide which can be used in the present invention is particularly chosen from the group consisting of tetramethoxysilane (TMOS, Si(OCH₃)₄), tetraethoxysilane (TEOS, Si(OC₂H₅)₄), tetrapropoxysilane (TPOS, Si(OC₃H₇)₄), tetrabutoxysilane (TBOS, Si(OC₄H₉)₄), trimethoxysilane (TMOS, HSi(OCH₃)₃), methyltrimethoxysilane [(CH₃)Si(OCH₃)₃], ethyltrimethoxysilane [(C₂H₅)Si(OCH₃)₃], propyltrimethoxysilane [(C₃H₇) Si (OCH₃)₃], vinyltrimethoxysilane [(C₂H₃)Si(OCH₃)₃], triethoxysilane [HSi(OC₂H₅)₃], methyltriethoxysilane [(CH₃)Si(OC₂H₅)₃], ethyltriethoxysilane [(C₂H₅)Si(OC₂H₅)₃], propyltriethoxysilane [(C₃H₇) Si (OC₂H₅)₃], vinyltriethoxysilane [(C₂H₃)Si(OC₂H₅)₃], and mixtures thereof.

Advantageously, the first silicon alkoxide used in the present invention is TMOS or TEOS.

More particularly, the first silicon alkoxide used in the present invention is TEOS. Indeed, with said precursor, it is possible, for the silica shell, to obtain a layer whose porosity is optimal to allow the molecule(s) of interest, such as DNA encapsulated in the core of the particle, to be accessible to constituents of large size present in the outside medium.

The second silicon alkoxide used in the present invention comprises at least one group capable of setting up an ionic and/or hydrogen non-covalent bond with the molecule of interest. This second alkoxide may have a hydrolysis rate equal to or higher than the hydrolysis rate of the first silicon alkoxide, thereby allowing the molecule(s) of interest to be concentrated in the core of the silica particle.

The absence of a said alkoxide prevents the encapsulation of the molecule of interest in the silica particle.

The second silicon alkoxide is advantageously of formulas R₇Si(OR₈)₃ or R₉R₁₀Si(OR₁₁)₂ where R₈ and R₁₁, the same or different, are an alkyl radical with 1 to 6 carbon atoms and R₇, R₉ and R₁₀, the same or different, are an alkyl radical with 1 to 8 carbon atoms, a heteroalkyl radical with 1 to 10 carbon atoms, an alkylaryl radical with 1 to 12 carbon atoms or an alkenyl radical with 1 to 8 carbon atoms,

the R₇ radical and at least one of the radicals R₉ and R₁₀ being substituted by at least one group capable of setting up an ionic and/or hydrogen non-covalent bond with the molecule of interest.

The radicals R₈ and R₁₁, the same or different, are an alkyl radical with 1 to 6 carbon atoms such as previously defined.

By [symbol] alkyl radical with 1 to 8 carbon atoms [symbol] is meant a straight-chain or branched alkyl radical having 1 to 8 carbon atoms, in particular 1 to 6 carbon atoms and more particularly 1 to 4 carbon atoms.

By [symbol] heteroalkyl radical with 1 to 10 carbon atoms [symbol] is meant a straight-chain or branched alkyl radical having 1 to 10 carbon atoms, in particular 1 to 8 carbon atoms, more particularly 1 to 6 carbon atoms and having at least one heteroatom such as N, S, O or P.

By [symbol] alkylaryl radical with 1 to 12 carbon atoms [symbol] is meant a straight-chain or branched alkyl radical having 1 to 12 carbon atoms, in particular 1 to 10 carbon atoms, more particularly 1 to 8 carbon atoms and further particularly 1 to 6 carbon atoms, having an aromatic or heteroaromatic substituent with 3 to 8 carbon atoms and optionally at least one heteroatom such as N, S, O or P.

By [symbol] alkenyl radical with 1 to 8 carbon atoms [symbol] is meant a straight-chain or branched alkenyl radical having at least one double bond and 1 to 8 carbon atoms, in particular 1 to 6 carbon atoms, more particularly 1 to 4 carbon atoms.

By [symbol] group capable of setting up an ionic and/or hydrogen non-covalent bond [symbol] in the present invention is meant a group chosen from the group consisting of —NH₂, —NHR₁₂ where R₁₂ is an alkyl radical with 1 to 6 carbon atoms such as previously defined, —NH₃⁺, —NH₂R₁₃⁺where R₁₃ is an alkyl radical with 1 to 6 carbon atoms such as previously defined, —COOH, —COO⁻, C(O)NH, —C(O), —SH and —OH. The group of the molecule of interest taking part in the non-covalent bond with the function in the core of the particle derived from the second silicon alkoxide may also comprise such a group.

Advantageously, the second silicon alkoxide which can be used in the present invention is chosen from the group consisting of N-trimethoxysilylpropyl-N,N,N-trimethylammonium chloride (C₉H₂₄ClNO₃Si, CAS: 35141-36-7); aminoethylaminomethyl)phenethyltrimethoxysilane (C₁₄H₂₆N₂O₃Si, CAS: 74113-77-2); N-(6-aminohexyl)aminopropyltrimethoxysilane (C₁₂H₃₀N₂O₃Si, CAS: 51895-58-0); 3-aminopropylmethyldiethoxysilane (C₈H₂₁NO₂Si, CAS: 3179-76-8); 3-aminopropyltrimethoxysilane (APTMES, C₆H₁₅NO₃Si, CAS: 13822-56-5); 3-aminopropyltriethoxysilane (APTES, C₉H₂₃NO Si, CAS: 919-30-2); 3-(2-aminoethylamino)propyltrimethoxysilane ((CH₃O₃Si (CH₂₃NHCH₂CH₂NH₂), CAS: 1760-24-3); (3-mercaptopropyl)trimethoxysilane (HS(CH₂)₃Si(OCH₃)₃, CAS: 4420-74-0), (3-mercaptopropyl)triethoxysilane (HS(CH₂)₃Si(OC₂H₅)₃, CAS: 14814-09-6) and mixtures thereof.

The second silicon alkoxide which can be used in the present invention is more particularly APTMES.

The silica particle of the invention is essentially obtained from hydrolysis of at least one first silicon alkoxide and at least one second silicon alkoxide such as previously defined. The groups capable of setting up an ionic and/or hydrogen non-covalent bond with the molecule of interest functionalizing the silica network of the particle are the result of the hydrolysis of the second silicon alkoxide.

The silica particle of the invention is essentially formed of units derived from the hydrolysis of at least one first silicon alkoxide and at least one second silicon alkoxide such as defined previously. The silica particle may comprise other elements, in particular at least one element capable of imparting magnetic properties thereto. Said elements capable of imparting magnetic properties are chosen in particular from the group consisting of iron, gadolinium, nickel, copper, chromium, cobalt, gold, silver, platinum, palladium, an oxide and a hydroxide thereof. For example the silica particle according to the invention, without the molecule(s) of interest, is composed of at least 80%, at least 90%, at least 95%, at least 98%, at least 99% of units derived from the hydrolysis of at least one first silicon alkoxide and at least one second silicon alkoxide such as previously defined. As a variant, the silica particle of the invention, without the molecule(s) of interest, is solely formed of units derived from the hydrolysis of first silicon alkoxide(s) and of second silicon alkoxide(s) such as previously defined.

The present invention also concerns a method for preparing a silica particle incorporating at least one molecule of interest according to the present invention. Any method for preparing such a particle from at least one first silicon alkoxide and at least one silicon alkoxide such as previously defined can be used in the present invention.

Advantageously, the present invention concerns a preparation method, in the presence of a molecule of interest, of at least one silica particle by reverse emulsion from:
at least one first silicon alkoxide (i.e. of formulas Si(OR₁)₄, R₂Si(OR₃)₃ or R₄R₅Si(OR₆)₂) such as previously defined,
at least a second silicon alkoxide having at least one group capable of setting up an ionic and/or hydrogen non-covalent bond with the molecule of interest such as previously defined.

By [symbol] reverse microemulsion [symbol] also called [symbol] water-in-oil [symbol] microemulsion is meant a limpid suspension, thermodynamically stable, of fine droplets of a first polar liquid in a second nonpolar liquid and hence non-miscible with the first liquid. The expressions [symbol] by reverse micellar route [symbol] and [symbol] via reverse microemulsion [symbol] are equivalent and can be used interchangeably.

The method according to the present invention may use:
one first silicon alkoxide such as previously defined and one second silicon alkoxide such as previously defined;
one first silicon alkoxide such as previously defined and several second silicon alkoxides such as previously defined;
several first silicon alkoxides such as previously defined and one second silicon alkoxide such as previously defined; or
several first silicon alkoxides such as previously defined and several second silicon alkoxides such as previously defined.

More particularly, the method of the invention comprises the following steps:

a) preparing a microemulsion (Mₐ) of water-in-oil type containing said molecule(s) of interest;

b) adding, to the microemulsion (Mₐ) prepared at step (a), a compound allowing the hydrolysis of a silicon alkoxide;

c) adding, to the microemulsion (M_{b}) obtained at step (b), at least one first silicon alkoxide such as previously defined and at least one second silicon alkoxide having at least one group capable of setting up an ionic and/or hydrogen non-covalent bond with the molecule of interest, such as previously defined;

d) adding to the microemulsion (M_{c}) obtained at step (c) a solvent allowing destabilization of the said microemulsion; and

e) recovering the silica particles incorporating at least one molecule of interest, precipitated at step (d).

Step (a) of the method of the invention therefore consists in preparing a microemulsion (Mₐ) of water-in-oil type containing at least one molecule of interest. Any technique allowing the preparation of such a microemulsion can be used in the present invention. It is therefore possible:
either to prepare a first solution (M₁) and subsequently incorporate therein one of more molecule(s) of interest to obtain the microemulsion (Mₐ);
or to prepare the microemulsion (Mₐ) directly by mixing together the different components and hence one or more molecule(s) of interest.

Advantageously, step (a) of the method of the invention consists in preparing a first solution (M₁) in which at least one molecule of interest is subsequently incorporated. This solution (M₁) is obtained by mixing together at least one surfactant, optionally at least one co-surfactant and at least one nonpolar or scarcely polar solvent. The surfactant, co-surfactant and nonpolar or scarcely polar solvent can be mixed at a single time or added one after the other or in groups. Advantageously, they are added one after the other and in the following order: surfactant, then optional co-surfactant, then nonpolar or scarcely polar solvent.

Mixing is conducted under agitation using an agitation, magnetic bar, ultrasound bath or homogenizer, and can be carried out at a temperature of between 10 and 40° C., advantageously between 15 and 30° C. and more particularly at ambient temperature (i.e. 23° C.±5° C.) for a time of between 1 min and 1 h, in particular between 10 and 45 min, more particularly between 15 and 30 min.

The surfactant(s) which can be used in the present invention are intended to introduce hydrophilic species into a hydrophobic environment and can be chosen from among ionic surfactants, non-ionic surfactants and mixtures thereof. By [symbol] mixtures [symbol] in the present invention is meant a mixture of at least two different ionic surfactants, a mixture of at least two different non-ionic surfactants or a mixture of at least one non-ionic surfactant and at least one ionic surfactant.

An ionic surfactant can notably be in the form of a charged hydrocarbon chain the charge of which is counter-balanced by a counter-ion. As non-limiting examples of ionic surfactants mention may be made of sodium bis(2-ethylhexyl)sulfosuccinate (AOT), cetyltrimethylammonium bromide (CTAB), cetylpyridinium bromide(CPB) and mixtures thereof.

A non-ionic surfactant which can be used in the present invention can be chosen from the group consisting of polyethoxylated alcohols, polyethoxylated phenols, oleates, laurates and mixtures thereof. As non-limiting examples of commercial non-ionic surfactants, mention may be made of the Triton X surfactants such as Triton X-100; Brij surfactants such as Brij-30; Igepal CO surfactants such as Igepal CO-520 or Igepal CO-720; Tweens such as Tween 20; Spans such as Span 85.

By [symbol] co-surfactant [symbol] in the present invention is meant an agent capable of facilitating the formation of microemulsions and stabilizing the same. Advantageously said co-surfactant is an amphiphilic compound chosen from the group consisting of a sodium alkyl sulfate with 8 to 20 carbon atoms such as SDS (Sodium Dodecyl Sulfate); an alcohol such as an isomer of propanol, butanol, pentanol and hexanol; a glycol and mixtures thereof. Advantageously, the co-surfactant, when it is present in solution (M₁), is n-hexanol.

Any nonpolar or scarcely polar solvent can be used in the present invention. Advantageously, the said nonpolar or scarcely polar solvent is an organic nonpolar or scarcely polar solvent and chosen in particular from the group consisting of n-butanol, hexanol, cyclopentane, pentane, cyclohexane, n-hexane, cycloheptane, heptane, n-octane, iso-octane, hexadecane, petroleum ether, benzene, isobutyl-benzene, toluene, xylene, cumenes, diethyl ether, n-butyl acetate, isopropyl myristate and mixtures thereof. Advantageously the nonpolar or scarcely polar solvent used in the present invention is cyclohexane.

In the solution (M₁), the surfactant is present in a proportion of between 1 and 40%, in particular between 5 and 30%, more particularly between 10 and 25% by volume relative to the total volume of said solution. The co-surfactant, when present in the solution (M₁), is in a proportion of between 1 and 30%, in particular between 5 and 25% and more particularly between 10 and 20% by volume relative to the total volume of said solution. Therefore, the nonpolar or scarcely polar solvent is present, in the solution (M₁), in a proportion of between 40 and 98%, in particular between 50 and 90% and more particularly between 60 and 80% by volume relative to the total volume of said solution.

Once the solution (M₁) is prepared, at least one molecule of interest such as previously defined is incorporated to form the microemulsion (Mₐ) of water-in-oil type.

The molecule(s) of interest can be added in solid form, in liquid form or in solution in a polar solvent. Irrespective of the variant implemented, a polar solvent is added to the microemulsion after the incorporation of the molecule(s) of interest in the solution (M₁). Advantageously, the molecule(s) of interest are added to the solution (M₁) in solution in a polar solvent, then a polar solvent the same or different from the first one is also added. More particularly, the two polar solvents used are the same. The adding of the molecule(s) of interest and optionally of the polar solvent can be conducted under agitation using an agitator, a magnetic bar, an ultrasound bath or a homogenizer.

By [symbol] polar solvent [symbol] in the present invention is meant a solvent chosen from the group consisting of water, deionized water, distilled water—acidified or basic, acetic acid, hydroxylated solvents such as methanol and ethanol, liquid glycols of low molecular weight such as ethyleneglycol, dimethylsulfoxide (DMSO), acetonitrile, acetone, tetrahydrofuran (THF) and mixtures thereof.

The polar solvent (polar solvent in which the molecule(s) of interest are in solution and/or other polar solvent subsequently added) is present in the microemulsion (Mₐ) to a proportion of between 0.25 and 20%, in particular between 0.5 and 10% and more particularly between 1 and 5% by volume relative to the total volume of said microemulsion. The molecule(s) of interest are present in this polar solvent in a quantity of between 0.05 and 10%, in particular between 0.1 and 5% more particularly between 0.2 and 1.5% by volume relative to the total volume of the polar solvent.

Step (b) of the method of the invention is intended to provide for hydrolysis of the silicon alkoxides by adding to the microemulsion (Mₐ) a compound allowing this hydrolysis; the microemulsion (M_{b}) thus obtained being a water-in-oil microemulsion.

By [symbol] compound allowing hydrolysis of a silicon alkoxide [symbol] in the present invention is meant a compound chosen from the group consisting of ammonia, sodium hydroxide (KOH), lithium hydroxide (LiOH) and sodium hydroxide (NaOH) and, advantageously, a solution of said compound in a polar solvent, the same or different from the polar solvent used at step (a). The compound allowing hydrolysis of a silicon alkoxide is more particularly ammonia or an ammonia solution in a polar solvent. Indeed, ammonia acts as reagent (H₂O) and as catalyst (NH₃) of the hydrolysis of a silicon alkoxide.

The compound chosen from the group consisting of ammonia, sodium hydroxide (KOH), lithium hydroxide (LiOH) and sodium hydroxide (NaOH), in solution in the polar solvent, is present in a proportion of between 5 and 50%, in particular between 10 and 40% and more particularly between 20 and 30% by volume relative to the total volume of the said solution. In addition, the said solution is present in a proportion of between 0.05 and 20%, in particular between 0.1 and 10% and more particularly between 0.2 and 5% by volume relative to the total volume of the microemulsion (M_{b}).

Step (b) can be carried out under agitation using an agitator, a magnetic bar, an ultrasound bath or a homogenizer at a temperature of between 10 and 40° C., advantageously between 15 and 30° C. and more particularly at ambient temperature (i.e. 23° C.±5° C.) for a time of between 5 and 45 min, in particular between 10 and 30 min and more particularly for 15 min.

Step (c) consists in incorporating in the microemulsion (M_{b}) thus obtained the silicon alkoxides such as previously defined which by sol-gel reaction will afford the silica of the silica particles according to the invention. The incorporation in the microemulsion (M_{b}) of the silicon alkoxides to obtain the microemulsion (M_{c}) of water-in-oil type is conducted under agitation using an agitator, a magnetic bar, an ultrasound bath or a homogenizer and can be carried out at a temperature of between 10 and 40° C., advantageously between 15 and 30° C. and more particularly at ambient temperature (i.e. 23° C.±5° C.) for a time of between 6 and 48 h, in particular between 12 and 36 h and, more particularly for 24 h.

The first silicon alkoxide(s) and the second silicon alkoxide(s) can be incorporated simultaneously in the microemulsion (M_{b}). Alternatively, they can be incorporated successively. In this case, the second silicon alkoxide(s) are advantageously incorporated before the first silicon alkoxide(s).

In the microemulsion (M_{c}) the silicon alkoxides i.e. the first+second silicon alkoxide(s) are present in a proportion of between 0.05 and 20%, in particular between 0.1 and 10% and more particularly between 0.5 and 5% by volume relative to the total volume of said microemulsion. Advantageously, the molar ratio of first silicon alkoxide(s)/second silicon alkoxide(s) is between 1:0.005 and 1:0.5; in particular between 1:0.01 and 1:0.1; and more particularly between 1:0.02 and 1:0.05.

Step (d) of the method of the invention is intended to precipitate the silica particles through the addition of a solvent which does not denature the structure of the nanoparticles but destabilizes or denatures the microemulsion (M_{c}) obtained at step (c).

Advantageously, the solvent used is a polar solvent such as previously defined. One particular polar solvent to be used at step (d) is chosen from the group consisting of ethanol, acetone and THF. Therefore the addition is made to the microemulsion (M_{c}) of a volume of solvent identical to or greater than the volume of said microemulsion, in particular greater by a factor of 1.2; more particularly greater by a factor of 1.5; even greater by a factor of 2 or 3.

Any technique allowing the recovery of silica particles incorporating at least one molecule of interest, precipitated at step (d) can be used at step (e) of the method of the invention. Advantageously, this step (e) entails one or more steps, the same or different, chosen from the steps of centrifugation, sedimentation and washings. The washing step(s) is(are) conducted in a polar solvent such as previously defined. If the recovery step entails several washings, one same polar solvent is used for several and even all the washings, or several different polar solvents are used for each washing. Concerning one or more centrifugation steps, these can be carried out by centrifuging the silica particles in particular in a washing solvent at ambient temperature, at a rate of between 4000 and 8000 rpm and in particular of the order of 5000 rpm (i.e. 5000±500 rpm) for a time of between 5 min and 2 h, in particular between 10 min and 1 h and more particularly for 15 min.

The method of the present invention may comprise an additional step, after step (e), intended to remove the free molecule(s) of interest and all traces of surfactant. Advantageously, this step consists in placing the silica particles recovered after step (e) in contact with a very large volume of water. By [symbol] very large volume [symbol] is meant a volume greater by a factor of 50, in particular by a factor of 500 and more particularly by a factor of 1000 than the volume of silica particles recovered after step (e) of the method of the invention. This step can be a dialysis step, the silica nanoparticles encapsulating one or more molecules of interest being separated from the volume by a cellulose membrane of Zellu Trans® type (marketed by Roth). Alternatively, it is possible to make provision for an ultrafiltration step instead of the dialysis step, via a membrane in polyethersulfone. This additional step may also be carried out under agitation using an agitator, a magnetic bar, an ultrasound bath or a homogenizer at a temperature of between 0 and 30° C., advantageously between 2 and 20° C. and more particularly under cold conditions (i.e. 6° C.±2° C.) for a time of between 3 h and 36 h, in particular between 6 h and 24 h and more particularly for 12 h.

For some applications of the particles according to the invention incorporating at least one molecule of interest, it may be necessary to concentrate these particles before re-suspending them in a suitable liquid or gel. Said concentration can be obtained, for liquids, by centrifugation. Another method known in biotechnology consists in preparing silica particles having magnetic properties. This objective can be achieved through the use of at least one element capable in imparting electromagnetic properties to the particle. This element may be iron oxide for example. In this case, the concentration and recovery of the silica particles according to the invention use a magnetic field.

Therefore, the method of the present invention may present a particular embodiment in which the silica particle that is prepared incorporating at least one molecule of interest is a silica particle comprising at least one element capable of imparting magnetic properties thereto such as a metallic constituent.

This embodiment comprises the addition to the microemulsion (Mₐ), to the micro-emulsion (M_{b}) and/or to the microemulsion (M_{c}) such as previously described at least one element capable of imparting magnetic properties to the silica particle (iron, gadolinium, nickel, copper, chromium, cobalt, gold, silver, platinum, palladium, or an oxide or hydroxide thereof) which is of sufficiently small size compared to the final size of the desired particle. In this variant, the condensation of the silica with the molecule(s) of interest by the second silicon alkoxide such as previously defined takes place by incorporating this element then, in similar fashion, a layer of silica by the first silicon alkoxide such as previously defined is created on the surface. Advantageously, this element is in the form of a magnetic particle.

The present invention also concerns the microemulsion (M_{c}) which can be used in the method of the invention. This microemulsion of water-in-oil type comprises:
at least one surfactant in particular such as previously defined;
optionally at least one co-surfactant in particular such as previously defined;
at least one nonpolar or scarcely polar solvent, in particular such as previously defined;
at least one polar solvent, in particular such as previously defined;
at least one molecule of interest, in particular such as previously defined;
at least one first silicon alkoxide, in particular such as previously defined;
at least one second silicon alkoxide having at least one group capable of setting up an ionic and/or hydrogen non-covalent bond with the molecule of interest, in particular such as previously defined;
at least one compound capable of hydrolysing said silicon alkoxides, in particular such as previously defined; and
optionally an element capable of imparting magnetic properties such as previously defined.

Advantageously the microemulsion of water-in-oil type, subject of the present invention comprises:
at least one surfactant in a quantity of between 1 and 40%, in particular between 5 and 30% and more particularly between 10 and 25%;
optionally at least one co-surfactant in a quantity of between 1 and 30%, in particular between 5 and 25% and more particularly between 10 and 20%;
at least one nonpolar or scarcely polar solvent in a quantity of between 40 and 95%, in particular between 50 and 90% and more particularly between 60 and 80%;
at least one polar solvent in a quantity of between 0.25 and 20%, in particular between 0.5 and 10% and in particular between 1 and 5%;
at least one molecule of interest in a quantity of between 0.0001 and 2%, in particular between 0.005 and 0.5% and more particularly between 0.001 and 0.1%;
at least one first silicon alkoxide in a quantity of between 0.05 and 20%, in particular between 0.1 and 10% and more particularly between 0.5 and 5%;
at least one second silicon alkoxide having at least one group capable of setting up an ionic and/or hydrogen non-covalent bond with the molecule of interest in a quantity of between 0.0005 and 0.2%, in particular between 0.001 and 0.1% and more particularly between 0.005 and 0.05%;
at least one compound capable of hydrolysing said silane-based compound in a quantity of between 0.01 and 5%, in particular between 0.05 and 1% and more particularly between 0.1 and 0.5%; and
optionally an element capable of imparting magnetic properties in a quantity of between 0.001 and 5%, in particular between 0.005 and 1% and more particularly between 0.01 and 0.5%;

the different quantities are expressed in volume relative to the total volume of the microemulsion.

The present invention finally concerns the use of a silica particle according to the invention or which can be prepared using a method of the invention, in different fields such as sensors, in vivo or in vitro diagnosis, traceability, combating infringement, the preserving and/or transport of molecules of interest.

Indeed, as explained in the foregoing, the silica particles of the invention have a certain number of characteristics listed below:
protection of the molecule of interest incorporated in the silica particle, and in particular in the core of the particle, by the silica;
holding the molecule of interest in the silica particle by means of non-covalent bonds between the molecule of interest and the units derived from the 2^{nd} silicon alkoxide;
porosity of the silica particle which allows the passing of molecules of small or large size.

In the application to sensors, the molecule of interest incorporated in the silica particle according to the invention is chosen so as to be capable of capturing a given element.

In the application to diagnosis, the silica particle of the invention, via the molecule of interest which it incorporated, can act as substrate for biological reactions. Therefore the present invention concerns the silica particle for use as diagnosis agent.

The experimental part below describes the use of silica particles incorporating a nucleic acid, and in particular a damaged DNA, to study the activity of repair enzymes or the reparatory activity of a given extract such as a cell extract. The silica particles of the invention can be placed in small volumes of biological extracts containing enzymes to be assayed. The silica particles can also be used in cellulo.

Alternatively, the inside of the silica particle according to the invention can act as reactor in which the reaction is triggered by the entry of a small substrate which passes through the pores of the silica (hybridization for example in blood circulation).

In applications to anti-infringement labelling and traceability, the molecule of interest is advantageously DNA since it allows the encoding of a large amount of information in a small volume, and this information can be amplified and decoded specifically by known, well-established protocols in biotechnology. DNA labelling has already been made available on the marketed by some companies. These solutions use DNA added in molecular form to liquids in particular.

In the present invention, the described experiments show that the DNA encapsulated in the silica particles remains accessible firstly for specific amplification (PCR) and secondly for in-situ repair. With this latter method, it is possible to obtain decoding of information by introducing a known damaged DNA into the particle followed by specific repair making it fluorescent and hence detectable. Having DNA inside a silica particle means that it can be sufficiently isolated from the ambient medium in which it is to be incorporated in order to obtain labelling (for applications to traceability or infringement detection). By encapsulating it in a silica particle it is also possible to provide access to functionalization of the silica so that these nanotracers are made compatible with varied solvents or materials. For example, it is possible to graft hydrophobic functions on the surface of the nanoparticles, such as thiol functions or carbon or fluorinated long chains and thereby to disperse these particles in organic solvents. This can be used for example for marking polymers or organic liquids.

Other characteristics and advantages of the present invention will become further apparent to those skilled in the art on reading the examples given below as non-limiting illustrations, with reference to the appended figures.

### BRIEF DESCRIPTION OF THE DRAWINGS

FIG. 1 gives a block diagram describing the structure of a silica nanoparticle with plasmidic DNA encapsulated in the core thereof, and the hydrogen interactions between the di-amino silica network and the phosphate groups of the DNA.

FIG. 2 shows transmission electron microscopy images of silica nanoparticles according to the invention encapsulating DNA.

FIG. 3 gives graphs showing the number distribution of the silica nanoparticles as a function of size, the silica nanoparticles having been synthesized following protocol II below: size about 40 nm (FIG. 3A) or following protocol I: size about 100 nm (FIG. 3B).

FIG. 4 gives a transmission electron microscopy image of silica nanoparticles according to the invention encapsulating polyacrylic acid (protocol IV).

FIG. 5 shows electrophoresis analysis on 1% agarose gel of non-functionalized silica nanoparticles synthesized with DNA (FIG. 5A) and silica nanoparticles according to the invention i.e. functionalized and synthesized with DNA (FIG. 5B). FIG. 5A: lanes 1 to 4 (control): markers of molecular weight, linearized plasmid (200 ng), supercoiled plasmid (200 ng) and silica nanoparticles respectively; lane 5: silica nanoparticles prepared in the presence of DNA then dialyzed; lanes 6 to 8: different dilutions of silica nanoparticles prepared in the presence of DNA. FIG. 5B: lanes 1 to 3 (control): markers of molecular weight, plasmid alone (14 ng, insufficient concentration), silica nanoparticles prepared without DNA; lane 4: empty; lane 5: silica nanoparticles encapsulating DNA and lane 6: silica nanoparticles encapsulating DNA-PI.

FIG. 6 shows fluorescent spectroscopy analyses of PI-labelled DNA in water (FIG. 6A), of silica nanoparticles encapsulating PI-labelled DNA in water (FIG. 6B), of Cy3-labelled DNA in water (FIG. 6C), and of silica nanoparticles encapsulating DNA labelled with Cy3 (FIG. 6D).

FIG. 7 gives emission spectra of nanoparticles analysed under confocal microscopy obtained after excitation at 488 nm. The studied nanoparticles are silica nanoparticles prepared without DNA (balls of pure silica), silica particles encapsulating DNA (silica/DNA balls), encapsulating DNA-PI (silica/DNA-PI balls) or encapsulating DNA-Cy3 (silica/DNA-Cy3 balls) subjected to excitation at 488 nm.

FIG. 8 illustrates the quantification of repair/nick-translation by fluorescence on 2% agarose gel, using Typhonn 9400 on DNA encapsulated in silica nanoparticles according to the invention, encapsulating DNA with dCTP-Cy3 as fluorescent marker, without enzyme [symbol] Blank [symbol] and with enzyme [symbol] Nick translation [symbol] (FIG. 8A) or with biotine-ddCTP subsequently detected by streptavidin-Fluoprobes 647, without enzyme [symbol] Blank [symbol] and with enzyme [symbol] Nick translation 4 [symbol] (FIG. 8B).

FIG. 9 shows the quantification of repair tests after gel electrophoresis on silica nanoparticles (Si) and on silica nanoparticles according to the invention encapsulating DNA (Si/DNA), damaged DNA (Si/damaged DNA), PAA and DNA (Si/PAA/DNA) or PAA and damaged DNA (Si/PAA/damaged DNA).

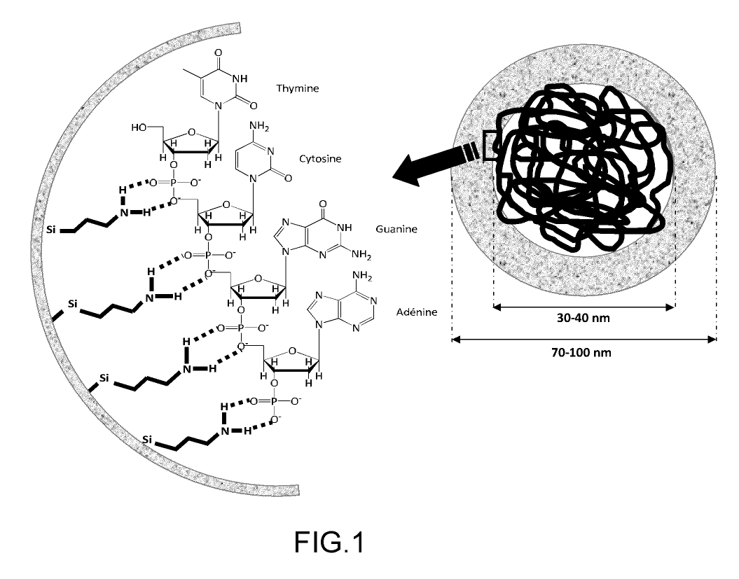

### DETAILED DESCRIPTION OF PARTICULAR EMBODIMENTS

I. Protocol for Synthesizing DNA-Encapsulating Nanoparticles

I.1. Nanoparticles Encapsulating DNA with Size of the Order of 100 nm (Protocol I)

The nanoparticles were prepared using the reverse microemulsion method (water-in-oil) [6].

The microemulsion solution was prepared by mixing the adequate quantities of surfactant, co-surfactant, organic solvent, water and aqueous ammonia solution, APTMES (3-aminopropyl-trimethoxysilane; d=1.027; M=179.29 gmol⁻¹) and TEOS (tetraethoxysilane; d=0.934; M=208.33 gmol⁻¹). The ammonia (NH₄OH) on decomposing acts as reagent (H₂O) and as catalyst (NH₃) for the hydrolysis of TEOS and of APTMES.

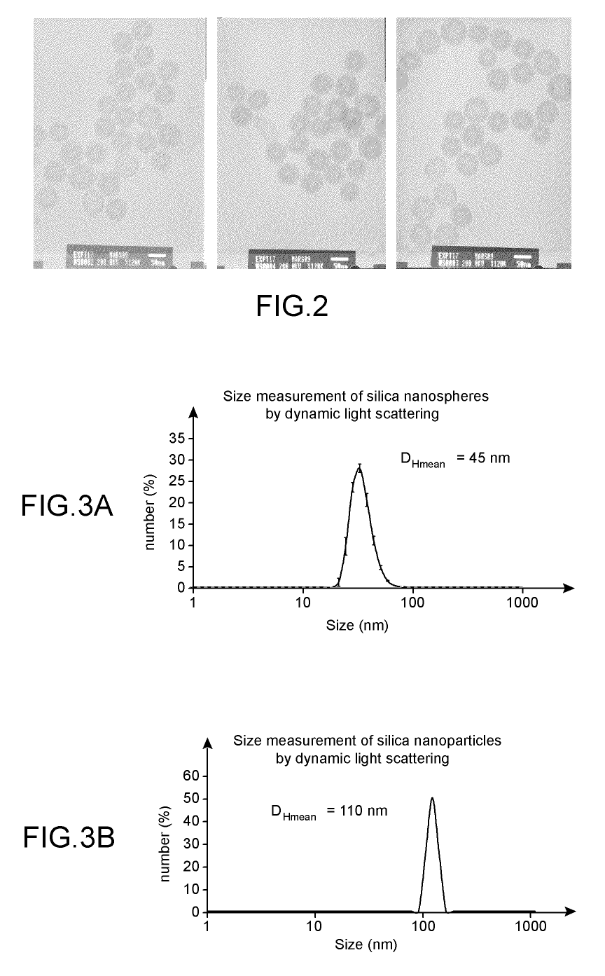

The conventional preparation of a reverse microemulsion for fabricating silica nanoparticles containing a plasmid is described below. This protocol allows particles to be obtained of the order of 100 nm (see TEM photos in FIG. 2).

The procedure consists in adding in a 50 mL flask, observing the described order, the following chemical products: triton X100 surfactant (2.1 mL), n-hexanol co-surfactant (2.05 mL), cyclohexane organic solvent (9.38 mL). The solution is then left under agitation at ambient temperature for 15 min.

Next, the plasmid (100 μL at 5 μg/μL in water, 3000 bp), water (200 μL) and 33% ammonia (125 μL, catalyst of the hydrolysis of the silicon alkoxides) are added to the solution. The formed emulsion is agitated for 15 min.

The silicon alkoxides, APTMES (1.5 μL) and TEOS (123.75 μL) are injected into this emulsion. The injection can be made successively starting with APTMES or else simultaneously. In both cases, the results obtained are identical. The reaction is left under agitation for 24 h at ambient temperature.

Finally, the emulsion is destabilized by adding ethanol (45 mL) and the nanoparticles are rinsed three times with ethanol and once with water. Each washing is followed by centrifugation at 5000 rpm for 15 min to sediment the nanoparticles. The silica nanoparticles are dispersed by vortexing in water (5 mL). They are then dialyzed overnight in 600 ml H₂O (MWCO: 4000-6000), allowing the removal of free DNA and any traces of surfactant. They are then ready to be characterized and used.

I.2. Nanoparticles Encapsulating DNA with a Size of the Order of 50 nm (Protocol II)

To obtain particles of 50 nm, the protocol is modified by changing the water/surfactant/oil phase ratio.

The procedure consists in adding in a 50 mL flask and in the given order the following chemical products: triton X100 surfactant (6.7 mL), n-hexanol co-surfactant (6.6 mL), cyclohexane organic solvent (15 mL). The solution is then left under agitation at ambient temperature for 15 min.

Next, the plasmid (100 μL at 5 μg/μL in water, 3000 bp), water (300 μL) and 33% ammonia (100 μL, catalyst of the hydrolysis of the silicon alkoxides) are added to the solution. The formed emulsion is left under agitation for 15 min.

The silicon alkoxides APTMES (1.5 μL) and TEOS (98.5 μL) are injected into this emulsion. The reaction is left under agitation for 24 h at ambient temperature.

Finally, the emulsion is destabilized through the addition of ethanol (45 mL) and the nanoparticles are rinsed three times with ethanol and once with water. Each washing is followed by centrifugation at 5000 rpm for 15 min to sediment the nanoparticles. The silica nanoparticles are dispersed by vortexing in water (5 mL). They are then dialyzed overnight in 600 ml H₂O (MWCO: 4000-6000), allowing the removal of small residual molecules. They are then ready for characterization and use.

I.3. Nanoparticles Encapsulating DNA with a Size of the Order of 15 nm (Protocol III)

To obtain even smaller particles (of the order of 15 nm) it is possible to use another surfactant following the protocol given below.

In a flask are mixed under magnetic agitation 1.3 mL of IGEPAL C0-520 surfactant with 10 mL of cyclohexane. Mixing is conducted at ambient temperature for 30 min.

A plasmid solution is then added (100 μL at 5 μg/μL in water) followed by a volume of 380 μL of water. A volume of 100 μL 33% ammonia is then added to the emulsion still under agitation.

The solution is left under agitation for 30 min to stabilize the system before successive or simultaneous adding of the silica precursors in the same manner as for the other protocols, namely: 1.5 μL APTMES and 98.5 μL of TEOS. If addition is made in succession, first APTMES is added followed by TEOS. The solution is left under agitation for 24 h.

Finally, the emulsion is destabilized through the addition of ethanol (45 mL) and the nanoparticles are rinsed three times with ethanol and once with water. Each washing is followed by centrifugation at 5000 rpm for 15 min to sediment the nanoparticles. The silica nanoparticles are dispersed by vortexing in water (5 mL). They are then dialyzed overnight in 600 ml H₂O (MWCO: 4000-6000), which allows the removal of free DNA and small residual molecules. They are then ready to be characterized and used.

However, in this case, if it is desired to encapsulate DNA, it is necessary to use a smaller plasmid (<3000 bp) even DNA strands. The final size of the nanoparticles also determines the size of the molecules that it is desired to encapsulate.

II. Protocol for Synthesizing Nanoparticles Encapsulating Polyacrylic Acid (Protocole IV)

The nanoparticles were prepared following protocol I. The only difference lies in the step in which the polyacrylic acid polymer (100 μL at 5 μg/μL in water i.e. a concentration of 0.4 μM, MW=1 250 000 gmol⁻¹) is added to the synthesis instead of DNA.

III. Protocol for Synthesizing Nanoparticles Encapsulating Both DNA and Polyacrylic Acid (Protocol V)

In a flask are mixed under magnetic agitation 2.1 mL of Triton X100 surfactant with 2.05 mL of n-hexanol and 9 mL of cyclohexane. Mixing is performed at ambient temperature for 15 min.

50 μL of polyacrylic acid solution are then added (MW=1 250 000 gmol⁻¹, C=0.4 μM) and a plasmid solution (100 μL at 5 μg/μL in water, 3000 bp) followed by a volume of 150 μL of water.

Next a volume of 125 μL of 33% ammonia is added to the emulsion still under agitation. The solution is left under agitation for 30 min to stabilize the system before the successive or simultaneous addition of the silica precursors in the same manner as for the other protocols, namely: 1.5 μL APTMES and 123.6 μL TEOS. If addition is made in succession first APTMES is added then the TEOS. The solution is left under agitation for 24 h.

The emulsion is finally destabilized through the addition of ethanol (45 mL) and the nanoparticles are rinsed tree times with ethanol and once with water. Each washing is followed by centrifugation at 5000 rpm for 15 min to sediment the nanoparticles. The silica nanoparticles dispersed by vortexing in water (5 mL) are dialyzed for 24 h. The dialyzed particles are then ready to be characterized and used (MWCO: 4000-6000).

The polyacrylic acid/DNA ratio can be modified to obtain more voluminous objects and having a thinner silica layer.

IV. Characterization of the Nanoparticles Encapsulating DNA and/or Polyacrylic Acid

The results and characterizations given below were obtained on silica nanoparticles in which plasmids were incorporated by reverse micellar synthesis. This characterization was able to show:
firstly the efficacy of the DNA encapsulating method; and
secondly the functionality of these objects i.e. the accessibility of the encapsulated DNA for several types of DNA-specific biological reactions, despite the fact that it is encapsulated in the nanoparticles. This is due to the fact that the shell of the particle is formed of porous silica.

IV.1. Morphological Characterization

i. Encapsulation of DNA

The TEM images of the silica particles with encapsulated DNA according to protocol I are given in FIG. 2. These images show that the size of the particles is between 50 and 80 nm.

In addition, a very distinct contrast can be seen between the core of the particle and the surface layer. This contrast is due to a difference in electronic density which clearly shows that the outer layer of the particles is essentially composed of silica and the core is mostly formed of DNA.

Measurement of the particles by DLS (Dynamic Light Scattering—Nanosizer by Malvern) confirmed the TEM observations (FIG. 3).

ii. Encapsulation of Polyacrylic Acid

To confirm this point, a similar experiment was conducted this time by encapsulating a polymer of similar structure to DNA and whose behaviour with respect to silicon alkoxides and sol-gel synthesis is similar to DNA (protocol IV). This polymer is polyacrylic acid. The synthesis protocol used was identical to the one used for DNA except that DNA was replaced by polyacrylic acid (cf. item II).

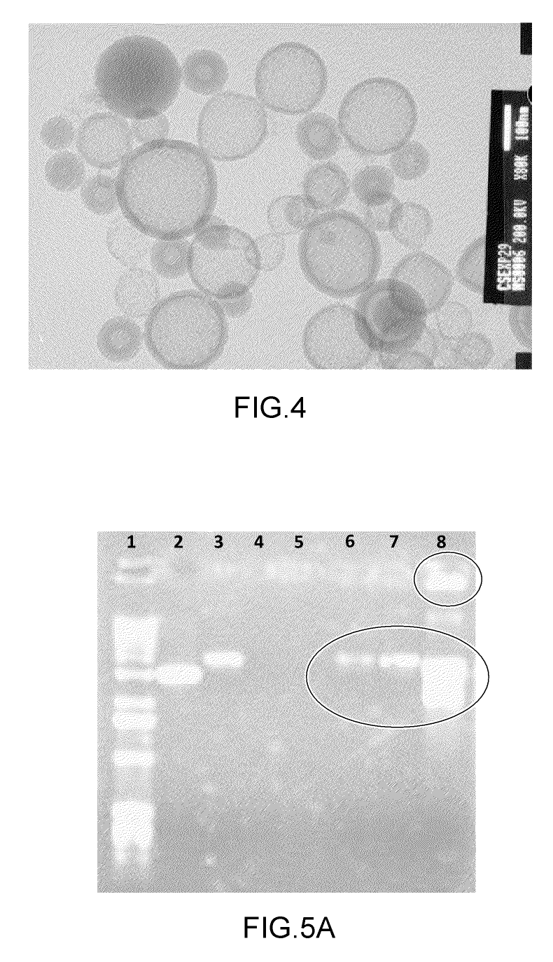

TEM characterization of the particles obtained shows similar structures with a less dense core than the shell. This characterization therefore shows that molecules such as DNA or polyacrylic acid are preferably found in the core of the particles and that a silica outer shell is formed (FIG. 4).

For polyacrylic acid, the size of the particles is much more polydispersed than for DNA, with particles ranging from 40 nm to 150 nm. This is probably due to perturbation of the emulsion by the polyacrylic acid badly dissolved in the aqueous phase. This dispersion is much less extensive with DNA encapsulation.

IV.2. Characterization of Surface Potential

To verify that the surface of the nanoparticles is indeed composed of silica and not DNA, the zeta potential was measured on balls with DNA and without DNA.

At pH 7, the potential of the silica particles was −30 mV, and that of the nanoparticles encapsulating DNA was −25 mV.

Since the surface potential of the particles from the two syntheses was practically identical, this measurement shows that the surface is mostly composed of silica and that the DNA is mostly confined to the core of the silica particle.

IV.3. Demonstration of the Presence of DNA in the Nanoparticles by Electrophoretic Method

To demonstrate that the previously described method using a silica precursor with amine functions (functionalized silica nanoparticles) allows the stable encapsulation of DNA in the nanoparticles, a comparative analysis was conducted of the migration profile on agarose gel of the different preparations in the presence of adapted controls.

With this method, a comparison is therefore made of the DNA trapping capacities of nanoparticles prepared using silica without any amine function (denoted Si) or with amine functions (called [symbol] functionalized [symbol] ; denoted SiNH₂) (Protocol I).

It is therefore possible to differentiate between the preparations in which DNA is trapped in the nanoparticles, and the preparations in which the DNA is merely adsorbed on the balls.

The following properties of electrophoresis on 1% agarose gel are used: the nanoparticles remain confined in the depositing well, the non-trapped DNA migrates in the gel under the force of the applied electric current. The DNA fragments are separated in relation to their molecular weight and their hindrance (1 kb has a molecular weight of 330 000 g.mol⁻¹). The negatively charged DNA migrates towards the anode whilst the positive ions of the buffer migrate towards the cathode thereby slowing the migration of the DNA by separating these fragments.

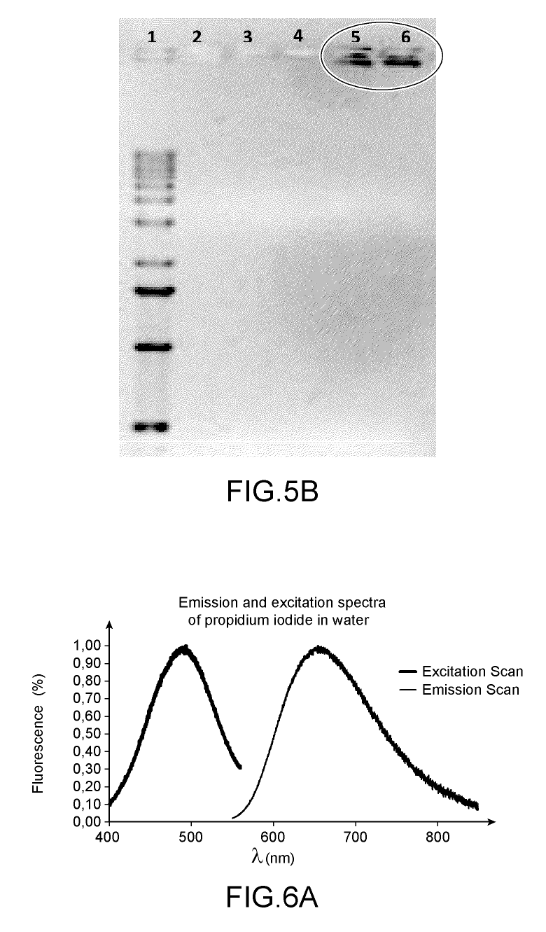

FIG. 5 shows the electrophoretic migration profile of the two preparations of nanoparticles/DNA in the presence of suitable controls. The DNA is detected by EtBr.

The silica nanoparticles deposited on the gel were synthesized from an emulsion containing DNA and only TEOS as silica precursor (denoted Si) (FIG. 5A). The silica balls deposited on the gel were synthesized with DNA, TEOS and a proportion of APTES following the protocol described previously (denoted Si/NH₂) (Protocol I) (FIG. 5B).

For the sample in FIG. 5A, the plasmids were encapsulated by adding them to the aqueous phase of the micellar synthesis. The electrophoretic profile on agarose gel of these nanoparticles was compared with the free non-encapsulated plasmids (lane 3) and with nanoparticles without DNA (lane 4). FIG. 5A shows the silica nanoparticles synthesized with plasmids before dialysis (lanes 6, 7 and 8) and after dialysis (lane 5) and nanoparticles of silica alone (lane 4) and non-encapsulated plasmids (lane 3).

This experiment shows that in lanes 6, 7 and 8, the plasmids which migrated such as the non-encapsulated reference plasmid (lane 3), are free. In addition, after dialysis (lane 5), the plasmids can no longer be seen in the gel, they were therefore removed during dialysis. It is concluded therefrom that the plasmids are pushed to the surface during synthesis of the nanoparticles and desorb during dialysis.

The results obtained with the nanoparticles synthesized following protocol I from the amine functions of APTES are given in FIG. 5B. The agarose gel shows that the nanoparticles co-localize with the plasmids detected by BET (lanes 5 and 6). These plasmids remain in the wells, there is no desorption during gel migration. This experiment indicates that by means of the functionalization, the plasmids remain attached to the nanoparticles. In addition, there is no separation by electrophoresis, the DNA is therefore located inside the nanoparticles trapped by the silica and the EtBr enters into the porous silica matrix to intercalate with the base pairs of the DNA.

IV.4. Demonstration of the Presence of DNA in the Nanoparticles by Confocal Microscopy and Spectrometry

Analyses by fluorescence and confocal microscopy were performed to confirm the presence of DNA in the functionalized nanoparticles. To detect DNA, it was first labelled with different fluorophores such as propidium iodide (PI; via simple incubation between the DNA and PI) and Cy3 (enzymatic labelling by nick-translation). These labelled plasmids were encapsulated using the method for functionalized nanoparticles (Protocol I). The results obtained are described below.

i. Fluorescence Analysis

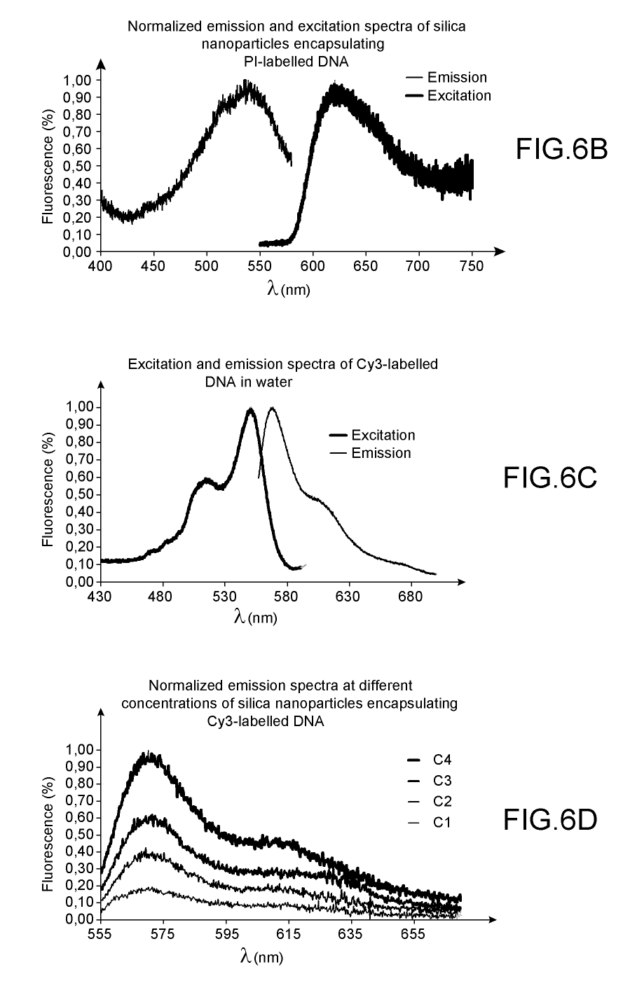

The graphs in FIGS. 6A and 6B allow a comparison between the fluorescence of PI in water (FIG. 6A) and of the silica nanoparticles with encapsulated DNA labelled with the PI intercalant (FIG. 6B). The same excitation and emission curves were observed in both cases. The DNA is therefore indeed present inside the silica particles.

The graph in FIG. 6C corresponds to analyses of the fluorescence of DNA labelled with Cy3 by nick-translation in water compared with that of nanoparticles encapsulating this same Cy3-labelled DNA (FIG. 6D). The graph in FIG. 6D shows the presence of the fluorescence emission peak of Cy3 in the silica nanoparticles. The DNA is therefore indeed present in the silica nanoparticles.

ii. Analysis by Confocal Microscopy

According to the confocal microscopy images, the nanoparticles not containing any labelled DNA are not visible (balls+PI alone, balls+non-labelled DNA, balls alone), unlike those which were prepared with fluorescent-labelled DNA.

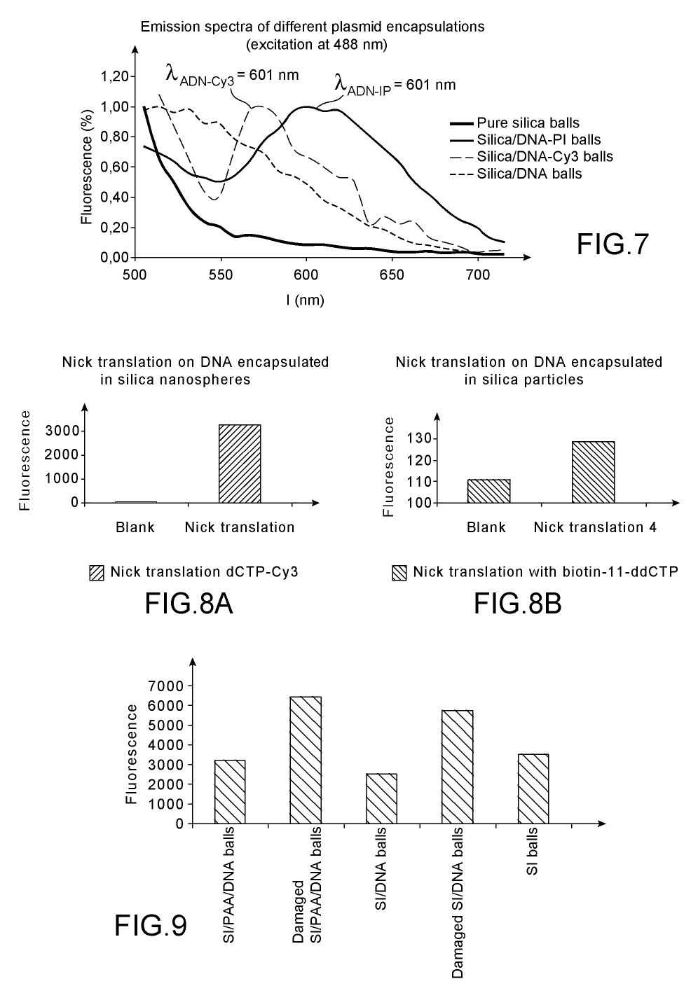

Polyvinyl acid films (PVA) were prepared and the studied nanoparticles were incorporated in these films to examine their fluorescence under confocal microscopy. The emission spectra of these nanoparticles are grouped together in FIG. 7. These curves tally with those in FIG. 6, which confirms the presence of the fluorophores in the silica nanoparticles.

V. Demonstration of Enzyme Accessibility of the DNA Encapsulated in the Nanoparticles of the Invention

The presence of DNA and its accessibility were evidenced by biological reactions conducted specifically on DNA. This allowed the specific labelling of DNA and hence the detection thereof.

V.1. Labelling and Repair of the Encapsulated DNA by Nick-Translation

The preceding results show that the silica nanoparticles synthesized and functionalized by reverse micellar route are capable of retaining the plasmids in their core. These plasmids remain accessible to small molecules of EtBr type which diffuse through the pores of the silica network.

The purpose of the following experiments was to show that the encapsulated DNA is also accessible to voluminous molecules of enzyme type which use DNA as substrate, in the enzymatic meaning of the term.

Enzymatic labelling was carried out by incubating the nanoparticles/DNA (Protocol I) with commercial enzymes and different labelled nucleotides. A control reaction (nanoparticles in the same reaction mixture but free of enzyme called a [symbol] blank [symbol] ) was conducted in parallel.

The method well-known to biologists of labelling by nick-translation followed using a commercial kit (N5500, Amersham, GE Healthcare) in the presence of dCTP-Cy3 or ddCTP-biotin. In this latter case, additional incubation in the presence of streptavidin-FluoProbes 647 (Interchim) is needed to detect the incorporation of ddCTP-biotin at DNA level.

### Experimental Protocol

The balls (40 μl) are incubated with 21 μl of each of dATP, dGTP, dTTP of the kit (300 μM solutions). The addition is made of 6 μl dCTP (300 μM), 3 μl dCTP-Cy3 or 3 μl ddCTP-biotin (Perkin Elmer), at 1 mM, and 30 μl of the kit enzyme mixture (containing a mixture of DNA polymerase I and DNAse I). The reaction takes for 4 h at 15° C. and is stopped thanks to through the addition of 6 μl 0.5 M EDTA. The balls are washed twice with 150 μl PBS containing 0.2 M NaCl and 0.1% Tween 20, then twice with distilled water.

If the marker used is ddCTP-biotin, 100 μl of this reaction are then incubated with 40 μl of streptavidin-FluoProbes 647 (Interchim) for 15 min at ambient temperature. The mixture is washed as described previously.

The nanoparticles are suspended in 100 μl of distilled water.

An identical quantity (10 μl) of the different nick-translation reactions is deposited in the wells loaded with 2% agarose gel.

To determine whether labelling by nick-translation has effectively taken place at the DNA, the fluorescence of the nanoparticles encapsulating the DNA is measured, after removal of the nucleotides non-incorporated in the DNA, by electrophoresis of the reaction mixtures on agarose gel. The labelling solutions are deposited in the loading wells of a 2% agarose gel. During electrophoresis, the nanoparticles remain in the deposit well, whilst the free DNA and the non-incorporated nucleotides, negatively charged, migrate in the agarose towards the anode. This can be particularly well seen in the lanes in which pure dCTP-Cy3 and dCTP-Cy5 were deposited, the Cy5 being equivalent to FluoProbes 657 in terms of fluorescence spectrum. The quantification of the fluorescence was made using a Typhoon 9400 reader (GE Healthcare). This apparatus allows the simultaneous quantification of several fluorophores.

The graph in FIG. 8A plots the quantification of the fluorescence present in the wells corresponding to the nick-translation test performed with dCTP-Cy3 as fluorescent marker, without enzyme [symbol] Blank [symbol] , and with the enzyme [symbol] Nick translation [symbol] .

The graph in FIG. 8B plots the quantification of the fluorescence present in the wells corresponding to the nick-translation test performed with biotin-ddCTP subsequently detected by streptavidin-Fluoprobes 647, without enzyme [symbol] Blank [symbol] , and with the enzyme [symbol] Nick translation 4 [symbol] .

It can be seen in the histograms that the fluorescence is stronger in the wells corresponding to the reactions performed in the presence of enzymes. This indicates that enzymatic labelling indeed took place at the DNA trapped in the nanoparticles and accessible to molecules in the medium outside the nanoparticles.

V.2. Test for Repair of Encapsulated DNA by Excision Re-Synthesis

During the DNA repair test, the nanoparticles prepared with or without plasmid DNA were incubated with active cell extracts (HeLa nuclear extracts), ATP and nucleotides labelled with a fluorophore.

The cell extract inter alia contained the enzymes responsible for DNA repair by excision re-synthesis (repair by base excision and repair by excision of nucleotides), ATP is essential for the catalysis of some enzymatic reactions and the fluorescent nucleotides are incorporated in the DNA by the polymerases contained in the extracts if repair takes place.

This experiment was conducted with nanoparticles/DNA prepared following Protocol I, using non-damaged DNA and damaged DNA.

This experiment was also performed using nanoparticles/DNA prepared following Protocol V (polyacrylic acid/DNA mixture).

The damaged DNA i.e. comprising base lesions was obtained by UVC radiation at a dose of 4.5 J/cm² of the non-damaged plasmids. The repair reaction was also performed using different balls prepared without DNA.

To conduct a repair test of DNA, the different components are added in adequate proportions. To a tube of final volume 50 μl are added ATG 5× buffer (200 mM Hepes KOH pH 7.8; 35 mM MgCl₂; 2.5 mM DTT; 1.25 μM dATP; 1.25 μM dTTP; 1.25 μM dGTP; 10 mM EDTA; 17% glycerol; 50 mM phosphocreatine; 250 μg/ml creatine phosphokinase; 0.5 mg/ml BSA), ATP (0.5 μl, 100 mM solution), dCTP-Cy5 (0.25 μl of 10⁵ M solution), HeLa nuclear extract (1 μl at 10 mg/ml; CilBiotech, Belgium), water and the nanoparticles dispersed in water. These are incubated for 4 h at 37° C. After the reaction, the nanoparticles are centrifuged and washed twice with 150 μl PBS containing 0.2 M NaCl and 0.1% Tween 20, once with ethanol then twice with distilled water. The nanoparticles are then dispersed in 100 μL of water.

An identical quantity (10 μl) of the different repair reactions was deposited in the loading wells of a 2% agarose gel.

Fluorescence measurement was carried out after removal of the nucleotides not incorporated in the DNA, by electrophoresis on agarose gel. During electrophoresis, the balls remain in the deposit well whist the free DNA and non-incorporated nucleotides migrate in the agarose towards the anode.

Quantification of the fluorescence in each well was performed using a Typhoon 9400 reader (GE Healthcare). The values are given in the histogram in FIG. 9.

In this histogram, the quantity of fluorescence is distinctly higher when the encapsulated DNA was previously damaged, than with undamaged DNA or no DNA at all. The repair reactions therefore indeed took place at the nanoparticles encapsulating the DNA. The encapsulated DNA is therefore indeed accessible to the enzymes contained in the outside medium and the reaction which took place was indeed specific.

The encapsulated DNA can therefore be used as substrate for the assay of activities of enzymes present in the outside medium.

### REFERENCES

[1] Stöber et al. (1968) [symbol] Controlled growth of monodisperse silica spheres in the micron size range [symbol] Journal of Colloid and Interface Science 26(1): 62-69.
[2] Bogush et al. (1988) [symbol] Preparation of monodisperse silica particles: Control of size and mass fraction [symbol] Journal of Non-Crystalline Solids 104(1): 95-106.
[3] Lindberg et al. (1997) [symbol] Multivariant analysis of the size dependence of monodisperse silica particles prepared according to the sol-gel technique [symbol] Colloids and Surfaces A: Physicochemical and Engineering Aspects 123-124: 549-560.
[4] Arriagada and Osseo-Asare (1999) [symbol] Synthesis of Nanosize Silica in a Nonionic Water-in-Oil Microemulsion: Effects of the Water/Surfactant Molar Ratio and Ammonia Concentration [symbol] Journal of colloid and interface science 211(2): 210-220.
[5] Yao et al. (2008) [symbol] The control of size and morphology of nanosized silica in Triton X-100 based reverse micelle [symbol] Colloids and Surfaces A: Physicochemical and Engineering Aspects 316(1-3): 8-14.
[6] Bagwe et al. (2004) [symbol] Optimization of dye-doped silica nanoparticles prepared using a reverse microemulsion method [symbol] Langmuir 20: 8336-8342.
[7] Lu et al. (2002) [symbol] Modifying the surface properties of superparamagnetic iron oxide nanoparticles through a sol-gel approach [symbol] Nanoletters, 2(3): 183-186.
[8] Wei et al. (2008) [symbol] Electrochemical and electrochemiluminescence study of Ru(bpy)²⁺₃-doped silica nanoparticles with covalently grafted biomacromolecules [symbol] Journal of Colloid and Interface Science 321: 310-314.
[9] Liu, et al. (2009) [symbol] Immobilization of trypsin on silica-coated fiberglass core in microchip for highly efficient proteolysis [symbol] Talanta 77: 1767-1773.
[10] Thi Phuong Binh Nguyen et al. (2008) [symbol] Synthesis of functionalized SBA-15 with ordered large pore size and its adsorption properties of BSA [symbol] Microporous and Mesoporous Materials 110(2-3): 560-569.
[11] Wu et al. (2006) [symbol] Surface properties of submicrometer silica spheres modified with aminopropyltriethoxysilane and phenyltriethoxysilane [symbol] Journal of Colloid and Interface Science 304: 119-124.
[12] Yoon et al. (2005) [symbol] Multifunctional nanoparticles possessing a “magnetic motor effect” for drug or gene delivery [symbol] Angew. Chem. Int. Ed. 44: 1068-1071.
[13] Samuel et al., (2008) [symbol] Dispersion and incorporation of optical nanotracers [symbol] , Proceeding of the Nanotech 2008, Boston.
[14] Fujiwara et al. (2007) [symbol] Direct encapsulation of BSA and DNA into silica microcapsules (hollow spheres) [symbol] J. Biomedical Mater. Res. Part A 81: 103-112.
[15] Tao et al. (2007) [symbol] The fabrication of hollow BSA micro/nanocapsules [symbol] J. Nanosci. Nanotechnol. 7(8): 2930-2932.
[16] He et al. (2007) [symbol] Preparation of luminescent Cy5 doped core-shell SFNPs and its application as near infrared fluorescent marker [symbol] Talanta 72: 1519-1526.
[17] Hammady et al. (2006) [symbol] Microemulsion and diafiltration approaches: an attempt to maximize the global yield of DNA-loaded nanospheres [symbol] European Journal of Pharmaceutics and Biopharmaceutics 62(2): 143-154.
[18] Cisse et al. (2007) [symbol] Fueling protein-DNA interactions inside porous nanocontainers [symbol] PNAS 104(31): 12646-12650.
[19] He et al. (2003) [symbol] Bioconjugated nanoparticles for DNA protection from cleavage [symbol] J. Am. Chem. Soc. 125: 7168-7169.
[20] Wang et al. (2008) [symbol] Fluorescent detection of ATP based on signaling DNA aptamer attached silica nanoparticles [symbol] Nanotechnology 19: 415605-415611.
[21] Gao et al. (2009) [symbol] Monodispersed mesoporous silica nanoparticles with very large pores for enhanced adsorption and release of DNA [symbol] J. Phys. Chem. B 113: 1796-1804.
[22] Pierre et al. (2001) [symbol] Encapsulation of deoxyribonucleic acid molecules in sica and hybrid organic-silica gels [symbol] J. Mat. Sci.: Mat. Med. 12: 51-55.

1.-18. (canceled)

19. A nanoparticle of porous silica incorporating at least one molecule of interest, wherein the silica network inside said nanoparticle is functionalized by at least one group capable of setting up an ionic and/or hydrogen non-covalent bond with the molecule of interest, whereby the molecule(s) of interest is(are) linked to the silica network solely by non-covalent bonds.

20. The silica nanoparticle according to claim 19, wherein the groups capable of setting up an ionic and/or hydrogen non-covalent bond with the molecule of interest functionalizing said nanoparticle are distributed within the latter in the form of a gradient decreasing from the centre of the nanoparticle towards the outside of the nanoparticle, no group being present on the surface of the nanoparticle.

21. The silica nanoparticle according to claim 19, wherein the silica nanoparticle is mesoporous with open porosity.

22. The silica nanoparticle according to claim 19, wherein said molecule of interest is selected from the group consisting of an enzyme, a protein, an oligopeptide, a peptide, an antigen, an antibody, a nucleic acid, a polymer and a carbohydrate.

23. The silica nanoparticle according to claim 19, wherein said group capable of setting up an ionic and/or hydrogen non-covalent bond is selected from the group consisting of: —NH₂, —NHR₁₂ where R₁₂ is an alkyl radical with 1 to 6 carbon atoms, —NH₃⁺, —NH₂R₁₃⁺ where R₁₃ is an alkyl radical with 1 to 6 carbon atoms, —COOH, —COO⁻, C(O)NH, —C(O), —SH and —OH.

24. The silica nanoparticle according to claim 19, wherein it comprises at least one element capable of imparting magnetic properties thereto.

25. A method for preparing a silica nanoparticle incorporating at least one molecule of interest according to claim 19, comprising:
preparing, in the presence of said molecule of interest, at least one silica particle by reverse emulsion, from:
at least one first silicon alkoxide of formulas Si(OR₁)₄, R₂Si(OR₃)₃ or R₄R₅Si(OR₆)₂ where R₁, R₃ and R₆, the same or different, are an alkyl radical with 1 to 6 carbon atoms and R₂, R₄ and R₅, the same or different, represent a hydrogen, an alkyl radical with 1 to 6 carbon atoms or an alkenyl radical with 1 to 6 carbon atoms, and
at least one second silicon alkoxide having at least one group capable of setting up an ionic and/or hydrogen non-covalent bond with the molecule of interest.

26. The method according to claim 25, wherein the radicals R₂, R₄ and R₅ of said first silicon alkoxide, the same or different, are selected from the group consisting of a hydrogen, methyl, ethyl, vinyl and propyl.

27. The method according to claim 25, wherein said first silicon alkoxide is selected from the group consisting of tetramethoxysilane, tetraethoxysilane, tetrapropoxysilane, tetrabutoxysilane, trimethoxysilane, methyltrimethoxysilane, ethyltrimethoxysilane, propyltrimethoxysilane, vinyltrimethoxysilane, triethoxysilane, methyltriethoxysilane, ethyltriethoxysilane, propyltriethoxysilane, vinyltriethoxysilane, and mixtures thereof.

28. The method according to claim 25, wherein the second silicon alkoxide is of formulas R₇Si(OR₈)₃ or R₉R₁₀Si(OR₁₁)₂ where R₈ and R₁₁, the same or different, are an alkyl radical with 1 to 6 carbon atoms, and R₇, R₉ and R₁₀, the same or different, are an alkyl radical with 1 to 8 carbon atoms, a heteroalkyl radical with 1 to 10 carbon atoms, an alkylaryl radical with 1 to 12 carbon atoms or an alkenyl radical with 1 to 8 carbon atoms,
the radical R₇ and at least one of the radicals R₉ and R₁₀ being substituted by at least one group capable of setting up an ionic and/or hydrogen non-covalent bond with the molecule of interest.

29. The silica nanoparticle according to claim 28, wherein said group capable of setting up an ionic and/or hydrogen non-covalent bond is selected from the group consisting of: —NH₂, —NHR₁₂ where R₁₂ is an alkyl radical with 1 to 6 carbon atoms, —NH₃⁺, —NH₂R₁₃⁺ where R₁₃ is an alkyl radical with 1 to 6 carbon atoms, —COOH, —COO⁻, C(O)NH, —C(O), —SH and —OH.

30. The method according to claim 25, wherein said second silicon alkoxide is selected from the group consisting of N-trimethoxysilylpropyl-N,N,N-trimethylammonium chloride, amino ethylaminomethyl)-phenethyltrimethoxysilane, N-(6-aminohexyl)aminopropyltrimethoxysilane, 3-aminopropyl-methyl-diethoxysilane, 3-aminopropyl-trimethoxysilane, 3-aminopropyl-triethoxysilane, 3-(2-aminoethylamino) propyl-trimethoxysilane, (3-mercaptopropyl)trimethoxysilane, (3-mercaptopropyl)triethoxysilane and mixtures thereof.

31. The method according to claim 25, wherein said method comprises the following steps:
a) preparing a microemulsion (Mₐ) of water-in-oil type containing said molecule(s) of interest;
b) adding, to the microemulsion (Mₐ) prepared at step (a), a compound allowing the hydrolysis of a silicon alkoxide;
c) adding, to the microemulsion (M_{b}) obtained at step (b), said at least one first silicon alkoxide and said at least one second silicon alkoxide having at least one group capable of setting up an ionic and/or hydrogen non-covalent bond with the molecule of interest;
d) adding, to the microemulsion (M_{c}) obtained at step (c) a solvent allowing the destabilization of said microemulsion; and
e) recovering the silica nanoparticles incorporating at least one molecule of interest precipitated at step (d).

32. The method according to claim 31, wherein said step (a) of the method consists in preparing a first solution (MO in which at least one molecule of interest is subsequently incorporated, said solution (MO being obtained by mixing together at least one surfactant, optionally at least one co-surfactant and at least one nonpolar or scarcely polar solvent.

33. The method according to claim 31, wherein at least one element capable of imparting magnetic properties to said silica particle is added to said microemulsion (Mₐ), to said microemulsion (M_{b}) and/or to said microemulsion (M_{c}).

34. A microemulsion of water-in-oil type (M_{c}) able to be used for a method according to claim 31, wherein it comprises:
at least one surfactant;
optionally at least one co-surfactant;
at least one nonpolar or scarcely polar solvent;
at least one polar solvent;
at least one molecule of interest;
at least one first silicon alkoxide;
at least one second silicon alkoxide having at least one group capable of setting up an ionic and/or hydrogen non-covalent bond with said molecule of interest;
at least one compound capable of hydrolysing said silicon alkoxides; and
optionally an element capable of imparting magnetic properties.

35. The microemulsion according to claim 34, wherein it comprises:
at least one surfactant in a quantity of between 1 and 40%, in particular between 5 and 30% and more particularly between 10 and 25%;
optionally at least one co-surfactant in a quantity of between 1 and 30%, in particular between 5 and 25% and more particularly between 10 and 20%;
at least one nonpolar or scarcely polar solvent in a quantity of between 40 and 95%, in particular between 50 and 90% and more particularly between 60 and 80%;
at least one polar solvent in a quantity of between 0.25 and 20%, in particular between 0.5 and 10% and more particularly between 1 and 5%;
at least one molecule of interest in a quantity of between 0.0001 and 2%, in particular between 0.005 and 0.5% and more particularly between 0.001 and 0.1%;
at least one first silicon alkoxide in a quantity of between 0.05 and 20%, in particular between 0.1 and 10% and more particularly between 0.5 and 5%;
at least one second silicon alkoxide having at least one group capable of setting up an ionic and/or hydrogen bond with said molecule of interest in a quantity of between 0.0005 and 0.2%, in particular between 0.001 and 0.1% and more particularly between 0.005 and 0.05%;
at least one compound capable of hydrolysing said silane-base compound in a quantity of between 0.01 and 5%, in particular between 0.05 and 1% and more particularly between 0.1 and 0.5%; and
optionally an element capable of imparting magnetic properties in a quantity of between 0.001 and 5%, in particular between 0.005 and 1% and more particularly between 0.01 and 0.5%;
the different quantities being expressed in volume relative to the total volume of the microemulsion.

36. A sensor comprising a silica nanoparticle according to claim 19.

37. A diagnosis agent comprising a silica nanoparticle according to claim 19.

38. An agent for traceability or for combating infringement comprising a silica nanoparticle according to claim 19.

39. A method for preserving or transporting molecules of interest consisting in incorporating said molecules of interest in a silica nanoparticle according to claim 19.
